(19) United States
(12) Patent Application Publication
(10) Pub. No.: US 2024/0161301 A1
(43) Pub. Date: May 16, 2024

(54) PREDICTING RESPONSE AND PROGNOSIS TO CDK 4/6 INHIBITORS BASED ON TUMOR VASCULARIZATION AND VESSEL SHAPE

(71) Applicant: Case Western Reserve University, Cleveland, OH (US)

(72) Inventors: Anant Madabhushi, Shaker Heights, OH (US); Nathaniel Braman, Cleveland, OH (US); Vidya Sankar Viswanathan, Shaker Heights, OH (US)

(21) Appl. No.: 18/500,473

(22) Filed: Nov. 2, 2023

Related U.S. Application Data
(60) Provisional application No. 63/425,028, filed on Nov. 14, 2022.

(51) Int. Cl. G06T 7/00; G06V 10/44; G16H 50/20

(52) U.S. Cl. CPC G06T 7/0016; G06V 10/44; G16H 50/20; G06T 2207/10116; G06T 2207/30056; G06T 2207/30068; G06T 2207/30096; G06T 2207/30101

(57) ABSTRACT

The present disclosure relates to a method. The method includes accessing data having one or more segmented images identifying one or more lesions and/or a plurality of blood vessels associated with the lesions. Respective ones of the blood vessels correspond to one or more centerlines and one or more constituent branches associated with the one or more lesions. The one or more segmented images are derived from one or more radiological images of a patient having cancer. One or more vascular radiology features are extracted using the centerlines, the constituent branches, and the one or more lesions. The one or more vascular radiology features relate to a quantification of the plurality of blood vessels or a shape of the plurality of blood vessels. The one or more vascular radiology features are used to determine a medical prediction associated with an outcome of the patient to cyclin-dependent kinase (CDK) inhibitor therapy.

### REFERENCE TO RELATED APPLICATION

This Application claims the benefit of U.S. Provisional Application No. 63/425,028, filed on Nov. 14, 2022, the contents of which are hereby incorporated by reference in their entirety.

### FEDERAL FUNDING NOTICE

This invention was made with government support under CA199374, CA202752, CA208236, CA216579, CA220581, CA239055, EB028736, and RR012463 awarded by the National Institutes of Health; W81XWH-19-1-0668, W81XWH-15-1-0558, and W81XWH-16-1-0329 awarded by the Department of Defense; and IBX004121A awarded by the Department of Veterans Affairs. The government has certain rights in the invention.

### BACKGROUND

Metastatic breast cancer (e.g., stage 4 breast cancer) is cancer that originated in the breasts, but that has spread to another part of the body (e.g., the liver, brain, bones, lungs, etc.). There are different sub-types of metastatic breast cancer, based on whether hormone receptors or other proteins are involved in how the cancer divides and grows. For example, metastatic breast cancer is classified as HR (hormone receptor) -positive if its cells have receptors for the hormones estrogen and progesterone, which suggests the cancer cells receive signals from these hormones that promote their growth.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates some embodiments of a block diagram corresponding to a cancer assessment system configured to use vascular radiology features extracted from a radiological image of an organ having one or more lesions to generate a medical prediction associated with an outcome of a patient.

FIG. 2 illustrates some embodiments of a flow diagram showing a method for using vascular radiology features extracted from a radiological image of an organ having one or more lesions to generate a medical prediction associated with an outcome of a patient.

FIG. 3 illustrates some additional embodiments of a block diagram corresponding to a disclosed cancer assessment system configured to use vascular radiology features to generate a medical prediction.

FIG. 4 illustrates some embodiments of a method of reducing blood vessels within a segmented image into centerlines and dividing a vasculature into constituent branches.

FIG. 5 illustrates some embodiments of a method of measuring a tortuosity of a blood vessel using a centerline.

FIGS. 6A-6B illustrate some embodiments of digitized images showing visualizations of a tumor vasculature in patients having different survival outlooks.

FIGS. 7A-7B illustrate some embodiments of Kaplan Meier survival curves generated according to the disclosed method and/or apparatus.

FIG. 8 illustrates some additional embodiments of a block diagram corresponding to a disclosed cancer assessment system configured to use vascular radiology features to generate a medical prediction.

FIG. 9 illustrates a table showing exemplary vascular radiology features that may be extracted during training of a disclosed cancer assessment system.

FIG. 10 illustrates some embodiments of a flow diagram showing a method for generating a machine learning model that uses vascular radiology features. computationally extracted from radiological images to generate a medical prediction.

FIG. 11 illustrates a block diagram of some embodiments of a prognostic apparatus comprising a disclosed cancer assessment system.

### DETAILED DESCRIPTION

The description herein is made with reference to the drawings, wherein like reference numerals are generally utilized to refer to like elements throughout, and wherein the various structures are not necessarily drawn to scale. In the following description, for purposes of explanation, numerous specific details are set forth in order to facilitate understanding. It may be evident, however, to one of ordinary skill in the art, that one or more aspects described herein may be practiced with a lesser degree of these specific details. In other instances, known structures and devices are shown in block diagram form to facilitate understanding.

Some types of breast cancer are affected by hormones, such as estrogen and/or progesterone. In such types of breast cancer, the cancer cells have receptors (proteins) that attach to hormones, which help them grow. Endocrine therapy is often used to treat patients having these types of breast cancer after surgery. Recently, endocrine therapy has begun to utilize cyclin-dependent kinases 4/6 (CDK 4/6) inhibitors to stop these hormones from attaching to breast cancer cell receptors and to thereby reduce a risk of the breast cancer returning.

CDK 4/6 affects cell division in humans. Its effect on cell division can drive cancer progression. For example, CDK 4/6 is believed to upregulate vascular endothelial growth factor (CEGF) and cause tortuous angiogenesis (e.g., the formation of new blood vessels), leading to a higher density of tortuous vasculature in aggressive tumors. CDK 4/6 inhibitors are thought to work by blocking cell cycles and cell proliferation by inhibiting CDK activity and thereby blocking the progression of cancerous cells. However, despite excellent efficacy in treating metastatic breast cancer (e.g., HR+ MBC, HER2− MBC, and/or the like) eventually all and/or nearly all patients become resistant to CDK 4/6 inhibitors thereby limiting its utility.

The present disclosure relates to a method and/or apparatus that uses vascular radiology features, which are computationally extracted from radiological images of a patient having received and/or receiving CDK 4/6 inhibitors, to make a medical prediction about an outcome of the patient. In some embodiments, the method comprises accessing an imaging data set comprising a radiological image of a patient that has received and/or that is receiving CDK 4/6 inhibitors for treatment of metastatic breast cancer. The radiological image is segmented to identify a lesion (e.g., metastasis) and a vasculature of blood vessels associated with the lesion. A plurality of vascular radiology features are extracted using the lesion and the vasculature of blood vessels. The plurality of vascular radiology features are subsequently used to generate a medical prediction associated with an outcome of the patient. Because of an association between CDK 4/6 inhibitors and tortuous angiogenesis, the vascular radiology features extracted from radiological images are able to provide information about the effect of CDK 4/6 in a patient. Therefore, the vascular radiology features can be used to generate a medical prediction relating to the patient (e.g., an efficacy of CDK 4/6 inhibitors, a survival of the patient, etc.) with a good accuracy that enables a level of care provided to metastatic breast cancer patients to be improved.

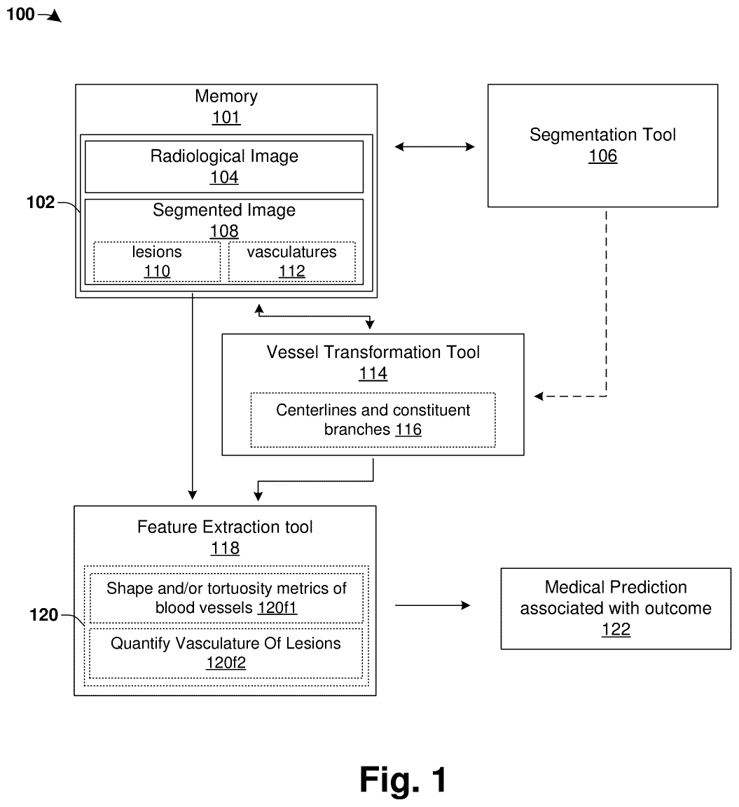

FIG. 1 illustrates some embodiments of a block diagram corresponding to a cancer assessment system 100 configured to use vascular radiology features extracted from a radiological image of an organ having one or more lesions to generate a medical prediction associated with an outcome of a patient.

The cancer assessment system 100 comprises a memory 101 configured to store an imaging data set 102 that includes one or more radiological images 104 of a patient that has or that has had cancer (e.g., hormone receptor positive (HR+) MBC, HER2− MBC, or the like). In some embodiments, the patient may have received and/or may be receiving cyclin-dependent kinases (CDK) inhibitor therapy (e.g., CDK 4/6 inhibitors). The one or more radiological images 104 may respectively comprise a digital image including an organ having one or more lesions (e.g., metastases). For example, the one or more radiology images 104 may comprise a digital image of one or more lesions within a liver of a patient.

A segmentation tool 106 may be in communication with the imaging data set 102. The segmentation tool 106 is configured to segment the one or more radiological images 104 to generate a segmented image 108 that identifies one or more lesions 110 and one or more vasculatures 112 (e.g., blood vessels) within the one or more radiological images 104. In some embodiments, the segmented image 108 may be stored in the memory 101. In other embodiments, the segmented image 108 may be provided directly from the segmentation tool 106 to a downstream tool.

A vessel transformation tool 114 is configured to reduce blood vessels within the segmented image 108 to centerlines and to divide a vasculature within the segmented image 108 into constituent branches. In some embodiments, the vessel transformation tool 114 may apply a fast marching algorithm to the vasculature of blood vessels to reduce the blood vessels to centerlines and to divide the vasculature into constituent branches.

A feature extraction tool 118 is configured to operate upon the centerlines and constituent branches 116 and/or the one or more lesions 110 to extract a plurality of vascular radiology features 120 describing the blood vessels and/or a vasculature associated with a lesion. In some embodiments, the plurality of vascular radiology features 120 may comprise metrics that describe a shape (e.g., a 3D shape) and/or tortuosity of the blood vessels 120f1 and/or metrics that quantify a vasculature associated with a lesion 120f2 (e.g., a number of blood vessels providing blood to and/or from a lesion or the like).

The cancer assessment system 100 is configured to utilize the plurality of vascular radiology features 120 to generate a medical prediction associated with an outcome of a patient 122. In some embodiments, the medical prediction associated with an outcome of a patient 122 may correspond to the patient's survival (e.g., a 5-year overall survival). In other embodiments, the medical prediction associated with an outcome of a patient 122 may correspond to the patient's response to treatment (e.g., a response to CDK 4/6 inhibitors). Because the tortuous vasculature of a tumor correlates to an aggressiveness of the tumor, the plurality of vascular radiology features 120 are able to be used to give the medical prediction associated with an outcome of a patient 122 a high accuracy.

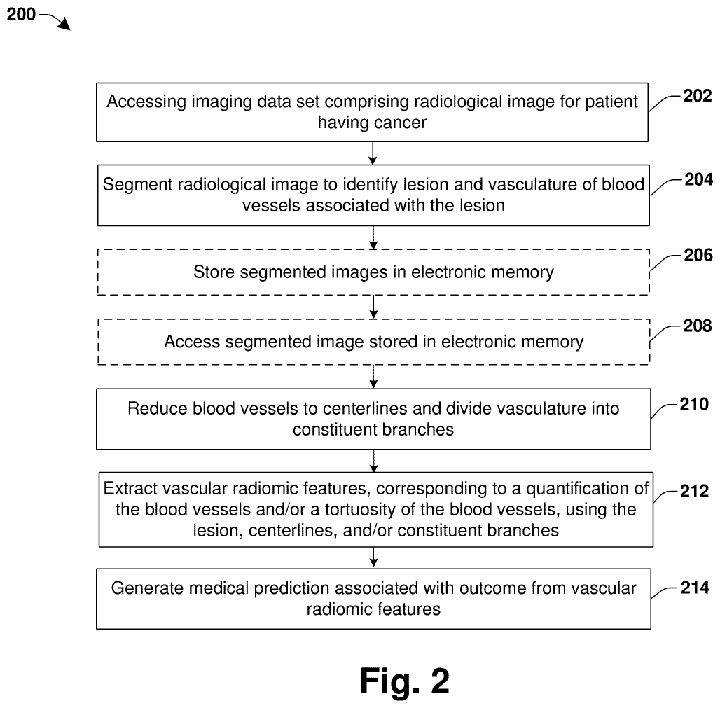

FIG. 2 illustrates some embodiments of a flow diagram showing a method 200 for using radiology features extracted from a radiological image of an organ having one or more lesions to generate a medical prediction associated with an outcome of a patient.

While the disclosed methods (e.g., methods 200 and 1000) are illustrated and described herein as a series of acts or events, it will be appreciated that the illustrated ordering of such acts or events are not to be interpreted in a limiting sense. For example, some acts may occur in different orders and/or concurrently with other acts or events apart from those illustrated and/or described herein. In addition, not all illustrated acts may be required to implement one or more aspects or embodiments of the description herein. Further, one or more of the acts depicted herein may be carried out in one or more separate acts and/or phases.

At act 202, an imaging data set is accessed, in some embodiments. In some embodiments, the imaging data set may comprise a radiological image from a patient being treated for cancer (e.g., MBC) with CDK inhibitors (e.g., CDK 4/6 inhibitors). The imaging data set may comprise one or more digitized images respectively including one or more lesions (e.g., lesions, tumors, etc.) within an organ (e.g., a liver, a brain, a lung) of the patient having cancer.

At act 204, the radiological image is segmented to form a segmented image that identifies a lesion and a vasculature of blood vessels associated with the lesion, in some embodiments. The segmentation may isolate the lesion and/or the vasculature of blood vessels associated with the lesion from other parts of the radiological image.

At act 206, the segmented image may be stored in electronic memory, in some embodiments.

At act 208, the segmented image may be accessed. Accessing the segmented image includes accessing data that comprises a segmented image identifying one or more lesions and a plurality of blood vessels associated with the one or more lesions

At act 210, the blood vessels within the segmented image are reduced to centerlines and the vasculature within the segmented image is divided into constituent branches. In some embodiments, a fast marching algorithm may be applied to the segmented image to reduce the blood vessels to centerlines and to divide the vasculature into constituent branches. In some embodiments, act 210 may be performed prior to act 208, so that within the segmented image that is accessed each blood vessel corresponds to one or more centerlines and one or more constituent branches associated with the one or more lesions.

At act 212, a plurality of vascular radiology features are extracted from the lesion, the centerlines, and/or the constituent branches. The plurality of vascular radiology features may relate to quantification of the blood vessels associated with the lesion (e.g., a number of and/or percentage of blood vessels arising from a lesion) and/or a tortuosity of the blood vessels.

At act 214, a medical prediction associated with an outcome of a patient (e.g., an overall survival and/or treatment response) may be generated using the plurality of vascular radiology features. A health care professional may make a decision as to the treatment of the patient based at least in part upon the medial prediction. For example, if the medical prediction is that the patient will stop responding to CDK 4/6 inhibitor treatment, the health care profession may choose to treat the patient with a surgical treatment (e.g., surgical recession of a lesion), to enroll the patient in a clinical trial, and/or the like. Therefore, the medical prediction can improve treatment of the patient so as to provide the patient with a better chance of survival and/or a higher quality of life.

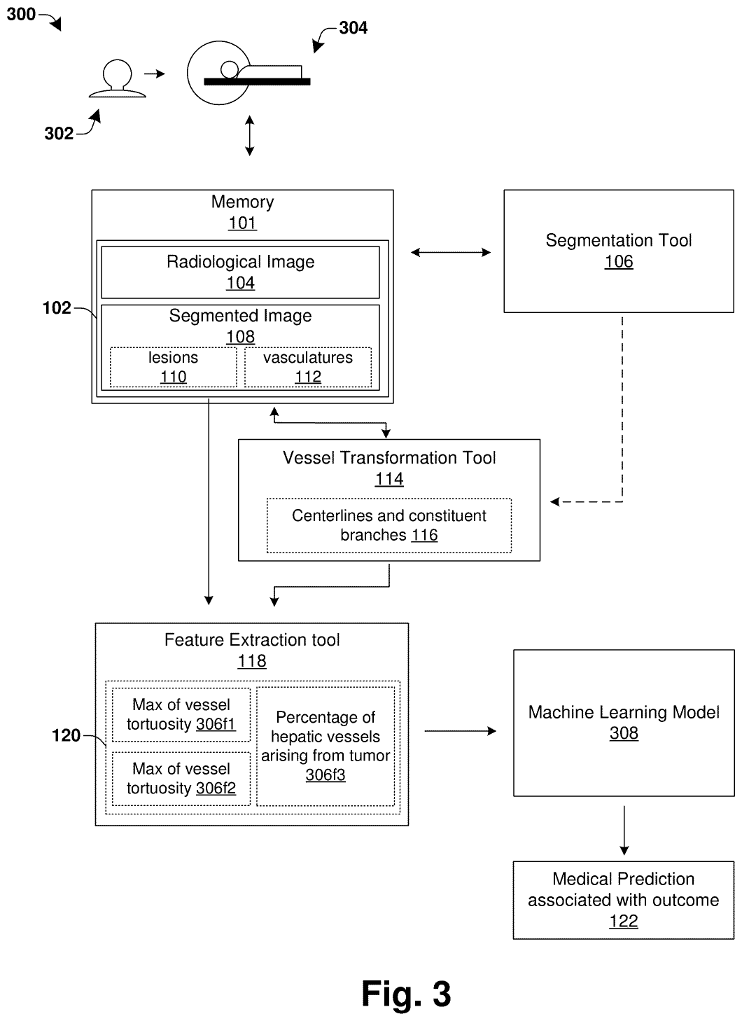

FIG. 3 illustrates some additional embodiments of a block diagram corresponding to a cancer assessment system 300 configured to use vascular radiology features extracted from a radiological image to generate a medical prediction associated with an outcome of a patient.

The cancer assessment system 300 comprises a memory 101 configured to store an imaging data set 102 that includes one or more radiological images 104 of a patient 302 that has or that has had cancer (e.g., hormone receptor positive (HR+) MBC, HER2− MBC, etc.). In some embodiments, the patient 302 may have received and/or may be receiving cyclin-dependent kinases 4/6 (CDK 4/6) inhibitors. In some embodiments, the memory 101 may comprise electronic memory (e.g., solid state memory, SRAM (static random-access memory), DRAM (dynamic random-access memory), and/or the like). In some embodiments, the one or more radiology images 104 within the imaging data set 102 may be obtained directly from one or more imaging tools 304 that are operated upon the patient 302. In various embodiments, the one or more radiology images 104 may comprise digital images from an x-ray, a computerized tomography (CT) scan, a magnetic resonance imaging (MRI) scan, nuclear imaging, positron emission tomography (PET) scan, a CT/PET scan, an ultrasound, or the like.

The one or more radiological images 104 may respectively comprise a digital image including an organ of the patient 302 having one or more lesions. For example, in some embodiments the one or more radiology images 104 may comprise a digital image of one or more liver metastases within a liver of the patient 302. In other embodiments, the one or more radiological images 104 may comprise a digital image of one or more metastases within a different organ (e.g., a brain, lungs, etc.) within the patient 302. In some embodiments, the one or more radiological images may comprise three-dimensional images.

In some embodiments, the one or more radiological images 104 may comprise images that are taken after treatment with CDK 4/6 inhibitors has begun. For example, the one or more radiological images 104 may comprise images taken after a first round of treatment with CDK 4/6 inhibitors has been performed (e.g., a first on-treatment image) and before a second round of treatment with CDK 4/6 inhibitors has been performed, images taken after a second round of treatment with CDK 4/6 inhibitors has been performed (e.g., a second on-treatment image) and before a third round of treatment with CDK 4/6 inhibitors has been performed, etc. While health care professionals may want patients to remain on CDK 4/6 inhibitors treatment as long as possible, some patients will become resistant to CDK 4/6 inhibitors treatment over time. By using radiological images that are taken after treatment with CDK 4/6 inhibitors has begun, the disclosed cancer assessment system 300 is able to account for a patient's response to the treatment with CDK 4/6 inhibitors, and thereby provide for an improved prediction of a patient's response to treatment with CDK 4/6 inhibitors (e.g., whether CDK 4/6 inhibitors treatment will remain effective to the patient). In some embodiments, the one or more radiological images 104 may comprise images taken from the patient 302 when the patient 302 is enrolled or considered for enrollment in a treatment plan that calls for further CDK 4/6 inhibitor treatments. In some embodiments, the one or more radiological images 104 may comprise images that are taken prior to treatment with CDK 4/6 inhibitors beginning.

A segmentation tool 106 may be configured to segment the one or more radiological images 104 to generate one or more segmented images 108 that identify one or more lesions 110 and one or more vasculatures 112 (e.g., blood vessels) within the one or more radiological images 104. In some embodiments, the segmentation tool 106 may comprise one or more volumetric masks (e.g., ternary masks having a first number associated with the one or more lesions 110, a second number associated with the one or more vasculatures 112, and a third number associated with other parts of the image). In some embodiments, the segmentation tool 106 may comprise a deep learning model configured to segment the radiological image to identify the one or more lesions and/or the one or more vasculatures.

A vessel transformation tool 114 is configured to reduce blood vessels within the segmented image 108 to centerlines and to divide a vasculature within the segmented image 108 into constituent branches. In some embodiments, the vessel transformation tool 114 may apply a fast marching algorithm to the segmented image 108 to reduce the blood vessels to centerlines and to divide the vasculature into constituent branches. In some embodiments, the vessel transformation tool 114 may be implemented as computer code run by a processing unit (e.g., a central processing unit including one or more transistor devices configured to operate computer code to achieve a result, a microcontroller, or the like).

A feature extraction tool 118 is configured to operate upon the centerlines and constituent branches 116 and/or the lesions 110 to extract a plurality of vascular radiology features 120. In some embodiments, the plurality of vascular radiology features 120 may comprise quantitative metrics computed by measuring a tortuosity and/or shape of a vascularity and/or a quantity of blood vessels associated with a lesion. Because CDK 4/6 is thought to act by upregulating VEGF (vascular endothelial growth factor) and thereby cause tortuous angiogenesis (e.g., the formation of new blood vessels from existing blood vessels) within a liver, such a plurality of vascular radiology features 120 are able to provide insight into an overall survival and/or treatment response for an associated patient. In some embodiments, the feature extraction tool 118 may be implemented as computer code run by a processing unit.

In some embodiments, the plurality of vascular radiology features 120 may comprise a vessel tortuosity generated by measuring a degree of twisting across a blood vessel separately for each of the constituent branches. The plurality of vascular radiology features 120 may further comprise one or more statistical measures/statistical assessments (e.g., a mean, standard deviation, maximum, skewness, kurtosis, and/or the like) of the tortuosity values over a patient level. In some additional embodiments, the plurality of vascular radiology features 120 may comprise a number and/or a percentage of vessels arising from a tumor. For example, plurality of vascular radiology features 120 may comprise a number and/or a percentage of hepatic blood vessels arising from a liver lesion. In some embodiments, plurality of vascular radiology features 120 may consist of a maximum of the vessel tortuosity computed for each branch 306f1, skewness of the vessel tortuosity computed for each branch 306f2, a percentage of hepatic blood vessels arising from the lesion 306f3.

A machine learning model 308 is configured to operate upon the plurality of vascular radiology features 120 to determine a medical prediction associated with an outcome of a patient 122. The medical prediction associated with an outcome of a patient 122 may comprise an overall survival and/or a treatment response of the patient 302. In some embodiments, the machine learning model 308 may comprise a regression model, such as a liner regression model, a Bayesian linear regression, a Cox proportional hazards model, or the like. In some embodiments, the machine learning model 308 may be implemented as computer code run by a processing unit.

In some embodiments, the feature extraction tool 118 may be configured to extract a first group of the plurality of vascular radiology features 120 from a first segmented image derived from a pre-treatment radiological image (e.g., a radiological image taken of the patient before initiation of treatment with CDK 4/6 inhibitor therapy) and to further extract a second group of the plurality of vascular radiology features 120 from a second segmented image derived from an on-treatment radiological image (e.g., a radiological image taken of the patient after initiation of treatment with CDK 4/6 inhibitor therapy). The machine learning model 308 is configured to generate the medical prediction associated with an outcome of a patient 122 using both the first group and the second group of the plurality of vascular radiology features 120 (e vascular radiology features extracted from the first segmented image derived from the pre-treatment radiological image and vascular radiology features extracted from the second segmented image derived from the on-treatment radiological image).

It will be appreciated that the disclosed methods and/or block diagrams may be implemented as computer-executable instructions, in some embodiments. Thus, in one example, a computer-readable storage device (e.g., a non-transitory computer-readable medium) may store computer executable instructions that if executed by a machine (e.g., computer, processor) cause the machine to perform the disclosed methods and/or block diagrams. While executable instructions associated with the disclosed methods and/or block diagrams are described as being stored on a computer-readable storage device, it is to be appreciated that executable instructions associated with other example disclosed methods and/or block diagrams described or claimed herein may also be stored on a computer-readable storage device.

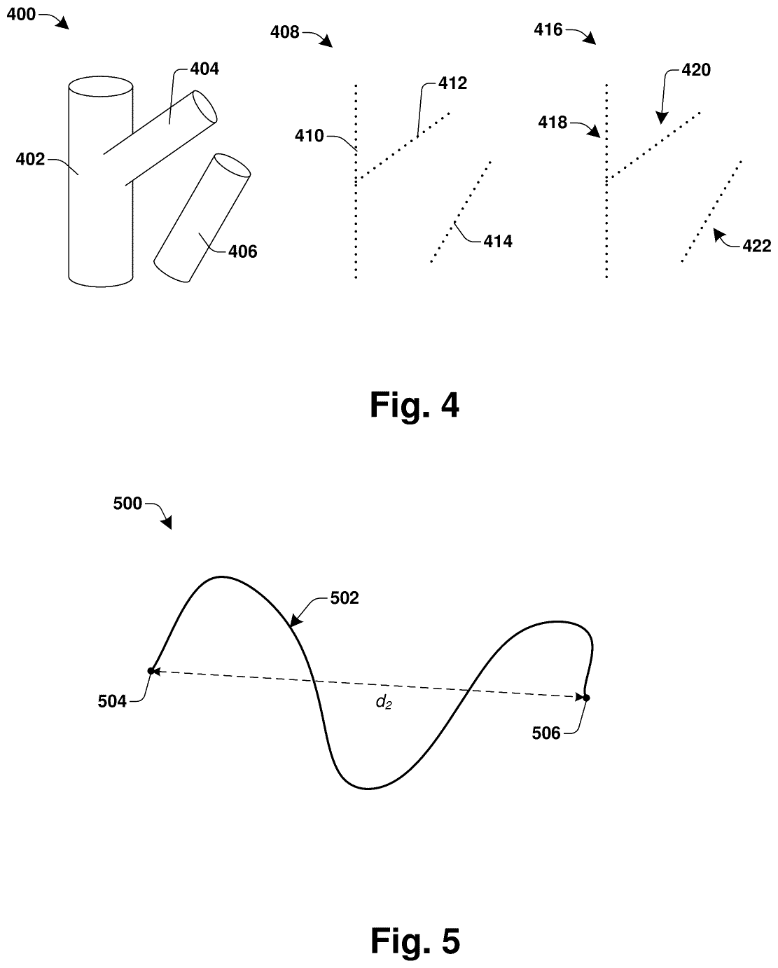

FIG. 4 illustrates some embodiments of a method of reducing blood vessels within a segmented image into centerlines and dividing a vasculature into constituent branches.

As shown in FIG. 4, a three-dimensional image of a vasculature 400 associated with a lesion comprises a first blood vessel 402, a second blood vessel 404 extending off of the first blood vessel 402, and a third blood vessel 406 that is separate from the first blood vessel 402 and the second blood vessel 404. In some embodiments, the vasculature 400 may be part of a segmented image.

A second image 408 shows the blood vessels 402-406 within the vasculature 400 represented as two-dimensional (2D) centerlines (e.g., lines that extend along centers of associated blood vessels). For example, the first blood vessel 402 is represented as a first centerline 410, the second blood vessel 404 is represented as a second centerline 412, and the third blood vessel 406 is represented as a third centerline 414. The centerlines 410-414 may be associated with a coordinate plane, so that different points of the centerlines 410-414 have different coordinates, thereby allowing for subsequent measurements to be taken on the blood vessels (e.g., centerlines).

A third image 416 shows the centerlines 410-414 of the vasculature divided into constituent branches 418-422. For example, the first centerline 410 is divided into a first branch 418, the second centerline 412 is divided into a second branch 420, and the third centerline 414 is divided into a third branch 422. Although the third image 416 shows each of the branches as having a single centerline, it will be appreciated that in other embodiments a branch may correspond to a group of two or more centerlines. The division of the vasculature into the constituent branches allows for the measurement of properties of individual vessel branches such as their shape (e.g., tortuosity). From this set of single-vessel measurements, statistics can be computed describing the variations in these properties across the tumor vasculature. In addition, division of the vessel network in the fashion allows for the quantification of features describing patterns of growth such as branching or the number or proportion of vessels feeding a tumor.

FIG. 5 illustrates some embodiments of an image 500 showing a method of measuring a tortuosity of a blood vessel using a centerline. Although FIG. 5 illustrates some examples of a method of measuring a tortuosity, it will be appreciated that the disclosure is not limited to such a method. Rather, in alternative embodiments, other methods of measuring a tortuosity (e.g., by measuring curvatures or the like) may be used.

As shown in image 500, a blood vessel is represented as a centerline 502 extending between a first point 504 and a second point 506. The centerline 502 winds up and down between the first point 504 and the second point 506. In some embodiments, a tortuosity of the blood vessel may be measured by measuring a first distance d₁ between the first point 504 and the second point 506 along the centerline 502. A second distance d₂ is also measured as a shortest distance between the first point 504 and the second point 506. The first distance d₁ and the second distance d₂ may be subsequently used to determine a tortuosity of the blood vessel. For example, the tortuosity may be calculated by subtracting the second distance d₂ divided by the first distance d₁ from 1 (i.e., tortuosity=1−(d₂/d₁). In such cases, a straight line will have tortuosity of 0 (since the first distance d₁ is equal to the second distance d₂), while a squiggly line will have a non-zero tortuosity (since the first distance d₁ is larger than the second distance d₂).

In some embodiments, respective blood vessels within an image (e.g., within a CT scan image) may be operated upon separately so as to separately measure a tortuosity for the individual blood vessels. For example, a first blood vessel may have a first tortuosity, a second blood vessel may have a second tortuosity, etc. Statistical assessments and/or measurements may be taken on the plurality of different tortuosities to provide for a patient level measurement of a tortuosity associated with the patient.

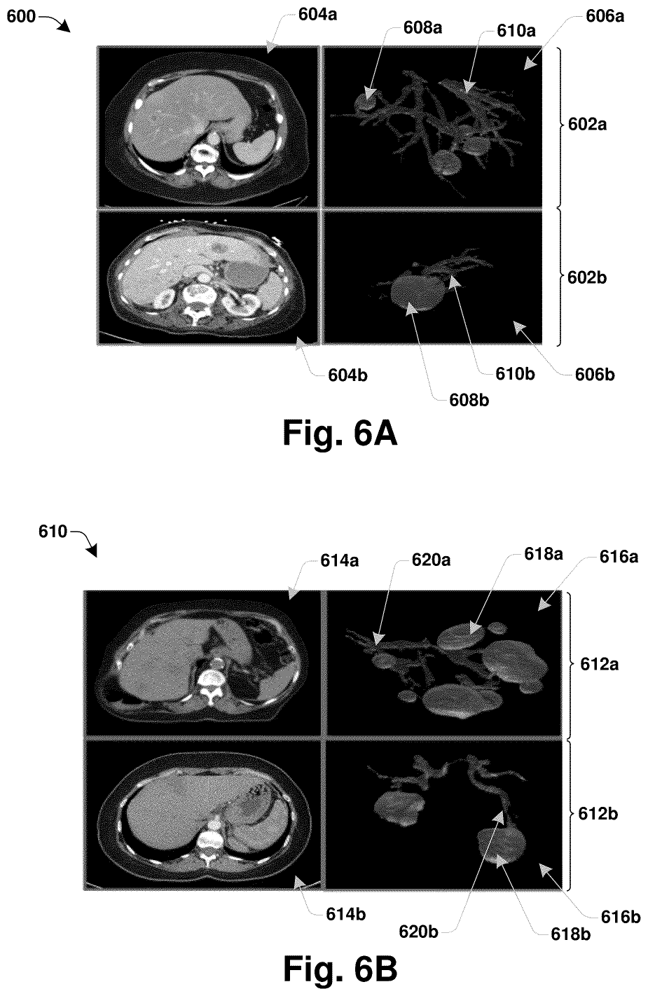

FIGS. 6A-6B illustrate some embodiments of digitized images showing visualizations of a tumor vasculature in patients having different survival outlooks.

FIG. 6A illustrates some embodiments of digitized images 600 for patients exhibiting a poor overall survival.

Digitized images 602a are associated with a first patient. Digitized images 602a show a CT scan image of a body cavity 604a having a liver with one or more liver lesions and a corresponding model 606a showing a lesion 608a and an associated vasculature 610a comprising blood vessels that are coupled to (e.g., providing blood to and/or from) the lesion 608a. The vasculature 610a shows a high percentage of blood vessels feeding the lesion 608a and a high level of vessel tortuosity.

Digitized images 602b are associated with a second patient. Digitized images 602b show a CT scan image of a body cavity 604b having a liver with one or more liver lesions and a corresponding model 606b showing a lesion 608b and an associated vasculature 610b comprising one or more blood vessels that are coupled to the lesion 608b. The vasculature 610b shows a high percentage of blood vessels feeding the lesion 608b and a high level of vessel tortuosity.

FIG. 6B illustrates some embodiments of digitized images 610 for patients exhibiting a good overall survival.

Digitized images 612a are associated with a third patient. Digitized images 612a show a CT scan image of a body cavity 614a having a liver with one or more liver lesions and a corresponding model 616a showing a lesion 618a and associated vasculature 620a comprising one or more blood vessels that are coupled to the tumor. In comparison to digitized images 600 of FIG. 6A, the vasculature 620a shows a relatively low density of blood vessels feeding the lesion 618a and a relatively low level of vessel tortuosity.

Digitized images 612b are associated with a fourth patient. Digitized images 612b show a CT scan image of a body cavity 614b having a liver with one or more liver lesions and a corresponding model 616b showing a lesion 618b and associated vasculature 620b comprising one or more blood vessels that are coupled to the tumor. In comparison to digitized images 600 of FIG. 6A, the vasculature 620b shows a relatively low density of blood vessels feeding the lesion 618b and a relatively low level of vessel tortuosity.

Therefore, from FIGS. 6A-6B it can be seen that higher numbers of blood vessels coupled to a tumor and/or higher levels of vessel tortuosity are associated with poor overall survival.

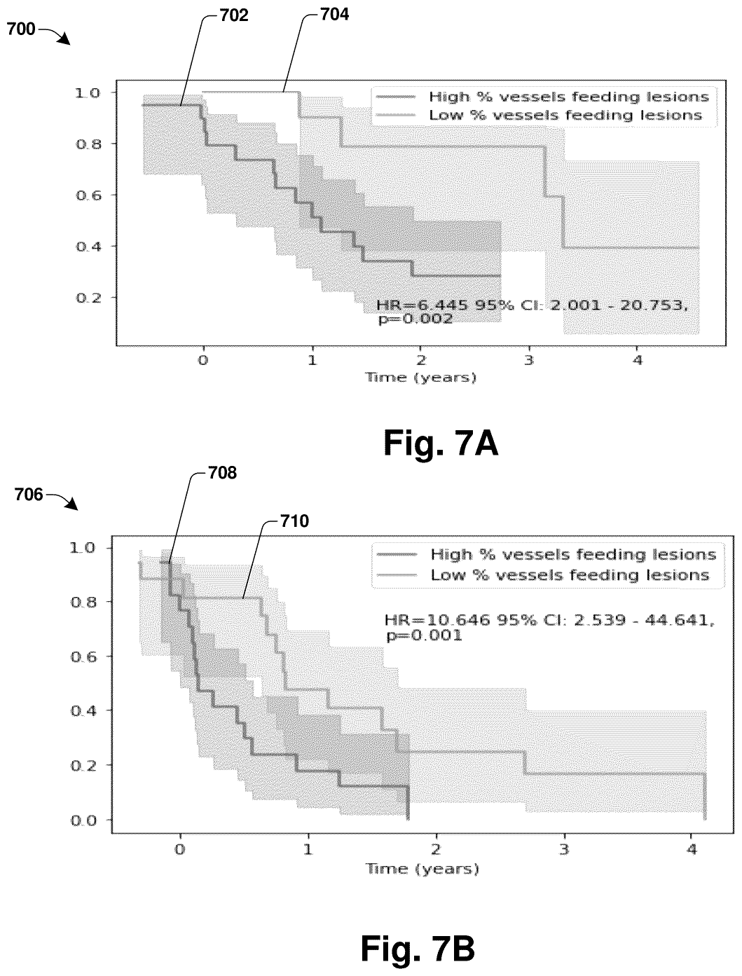

FIG. 7A illustrates some embodiments of a graph 700 showing Kaplan-Meier survival curves generated according to the disclosed method and/or apparatus. Graph 700 illustrates a survival time along an x-axis and a survival percentage on the y-axis.

Graph 700 illustrates a first Kaplan-Meier survival curve 702 and a second Kaplan-Meier survival curve 704. The first Kaplan-Meier survival curve 702 corresponds to a patient having a relatively high percentage of blood vessels feeding a lesion. The second Kaplan-Meier survival curve 704 corresponds to a patient having a relatively low percentage of blood vessels feeding a lesion (e.g., a relatively low percentage of blood vessels relative to a tumor associated with the first Kaplan-Meier survival curve 702). As can be seen in graph 700, the patient associated with the first Kaplan-Meier survival curve 702 has a significantly worse overall survival than the patient associated with the second Kaplan-Meier survival curve 704. Therefore, it can be seen from graph 700, that a percentage of blood vessels within a vasculature that are feeding a lesion is a vascular radiology feature that has a good correlation to overall survival.

FIG. 7B illustrates some additional embodiments of a graph 706 showing Kaplan-Meier survival curves generated according to the disclosed method and/or apparatus. Graph 706 illustrates a survival time along an x-axis and a survival percentage on the y-axis.

Graph 706 illustrates a third Kaplan-Meier survival curve 708 and a fourth Kaplan-Meier survival curve 710. The third Kaplan-Meier survival curve 708 corresponds to a patient having a relatively high percentage of blood vessels feeding a lesion. The fourth Kaplan-Meier survival curve 710 corresponds to a patient having a relatively low percentage of blood vessels feeding a lesion (e.g., a relatively low percentage of blood vessels relative to a tumor associated with the third Kaplan-Meier survival curve 708). As can be seen in graph 706, the patient associated with the third Kaplan-Meier survival curve 708 has a significantly worse overall survival than the patient associated with the fourth Kaplan-Meier survival curve 710. Therefore, graph 706 confirms that a percentage of blood vessels within a vasculature that are feeding a lesion is a vascular radiology feature that has a good correlation to overall survival.

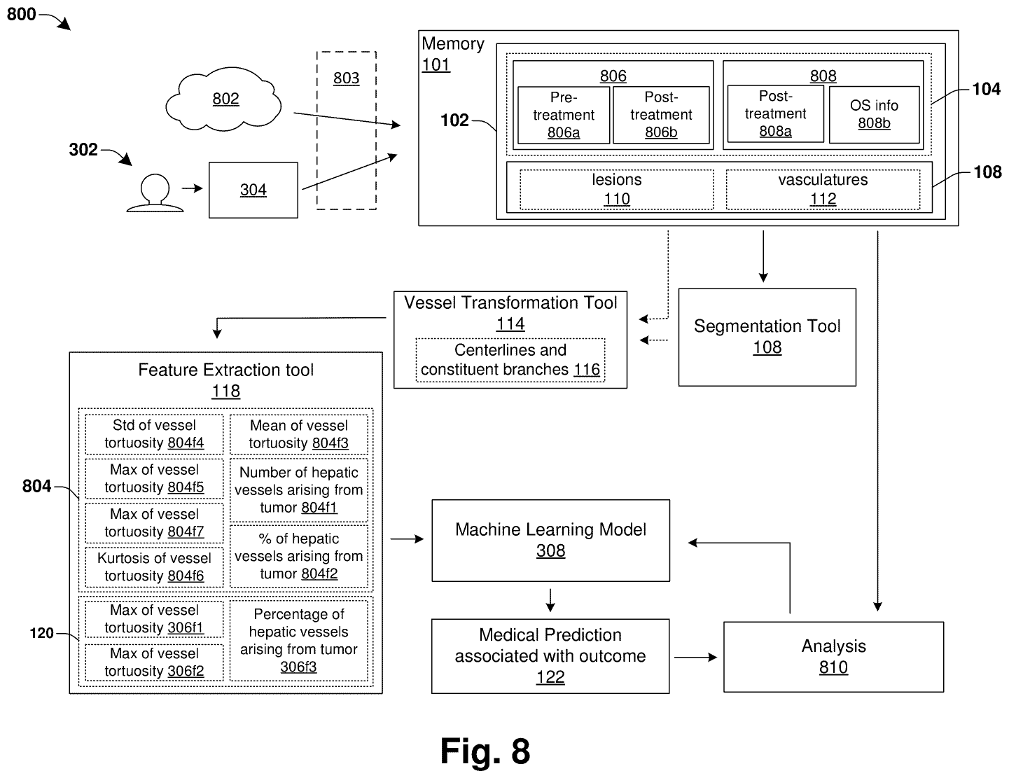

FIG. 8 illustrates some additional embodiments of a block diagram corresponding to a cancer assessment system 800 configured to use vascular radiology features extracted from radiological images to generate a medical prediction associated with an outcome of a patient.

The cancer assessment system 800 comprises a memory 101 configured to store an imaging data set 102 that includes one or more radiological images 104 of a patient that has or that has had cancer (e.g., MBC). In some embodiments, the patient may have received and/or may be receiving CDK 4/6 inhibitors. The one or more radiological images 104 may respectively comprise a digital image including an organ having one or more lesions. For example, the one or more radiology images 104 may comprise a digital image of one or more lesions within a liver of a patient. In some embodiments, the one or more radiology images 104 within the imaging data set 102 may be obtained directly from one or more imaging tools 304 (e.g., computerized tomography (CT) scan images from a CT scanner). In some additional embodiments, the one or more radiology images 104 within the imaging data set 102 may be obtained from an on-line database 802 and/or archive containing digitized pathology images from patients generated at different sites (e.g., different hospitals, research laboratories, and/or the like). Prior to including digitized pathology images within the imaging data set 102, the or more radiology images 104 may be subjected to image assessment 803 including one or more inclusion criteria and exclusion criteria.

In some embodiments, a segmentation tool 106 may be configured to segment the one or more radiological images 104 to generate one or more segmented images 108 that respectively identify one or more lesions 110 and one or more vasculatures 112 (e.g., blood vessels) within the one or more radiological images 104. A vessel transformation tool 114 is configured to reduce blood vessels within the segmented image 108 to centerlines and to divide a vasculature within the segmented image 108 into constituent branches. In some embodiments, the vessel transformation tool 114 may apply a fast marching algorithm to the vasculature of blood vessels to reduce the blood vessels to centerlines and to divide the vasculature into constituent branches.

A feature extraction tool 118 is configured to operate upon the centerlines and constituent branches 116 and/or the lesions 110 to extract a plurality of potential vascular radiology features 804. In some embodiments, the plurality of potential vascular radiology features 804 may comprise quantitative metrics computed by measuring a vascularity of lesions and a shape (e.g., a 3-D shape) and/or tortuosity of hepatic blood vessels. In some embodiments, the plurality of potential vascular radiology features 804 are respectively evaluated for an ability to determine a medical prediction associated with an outcome of a patient 122. Features of the plurality of potential vascular radiology features 804 that are found to prognostic are identified as a plurality of vascular radiology features 120.

In some embodiments, the plurality of potential vascular radiology features 804 may comprise seven radiology features consisting of a number of hepatic blood vessels arising from a lesion 804f1, a percentage of hepatic blood vessels arising from the lesion 804f2, a mean of a vessel tortuosity separately computed for each branch 804f3, a standard deviation of the vessel tortuosity computed for each branch 804f4, a maximum of the vessel tortuosity computed for each branch 804f5, skewness of the vessel tortuosity computed for each branch 804f6, and kurtosis of the vessel tortuosity computed for each branch 804f7. In some embodiments, plurality of vascular radiology features 120 may consist of a maximum of the vessel tortuosity computed for each branch 306f1, skewness of the vessel tortuosity computed for each branch 306f2, a percentage of hepatic blood vessels arising from the lesion 306f3.

In some embodiments, the one or more radiological images 104 may comprise a training set 806 and a test set 808. The training set 806 comprises radiological images from a first plurality of patients. The test set 808 comprises radiological images from a second plurality of patients. In some embodiments, the training set 806 may comprise both pre-treatment images 806a and on-treatment images 806b, while the test set 808 may comprise additional on-treatment images 808a, but not pre-treatment images. In some embodiments, the test set 808 may comprise additional on-treatment images 808a that have corresponding survival information 808b (e.g., overall survival data).

In some embodiments, the plurality of potential vascular radiology features 804 are evaluated using the pre-treatment images 806a within the training set 806 to identify a first set of features. The plurality of potential vascular radiology features 804 are further evaluated using the on-treatment images 806b within the training set 806 to identify a second set of features. In some embodiments, the pre-treatment images 806a and the on-treatment images 806b within the training set 806 may be evaluated for association with disease progression. The plurality of potential vascular radiology features 804 are further evaluated using the additional on-treatment images 808a within the test set 808 to identify a third set of features. A test medical prediction associated with an outcome of a patient may be generated based on the third set of features and then compared to the overall survival data to determine a validity of the third set of features. The first set of features, the second set of features, and the third set of features may be analyzed to determine the plurality of vascular radiology features 120. In some embodiments, the pre-treatment images 806a, the on-treatment images 806b, and the additional on-treatment images 808a may be evaluated using an analysis unit 810 including a Cox proportional hazards model. In some embodiments, the Cox proportional hazards model comprise a LASSO (least absolute shrinkage and selection operator) algorithm (e.g., a LASSO Cox regression model).

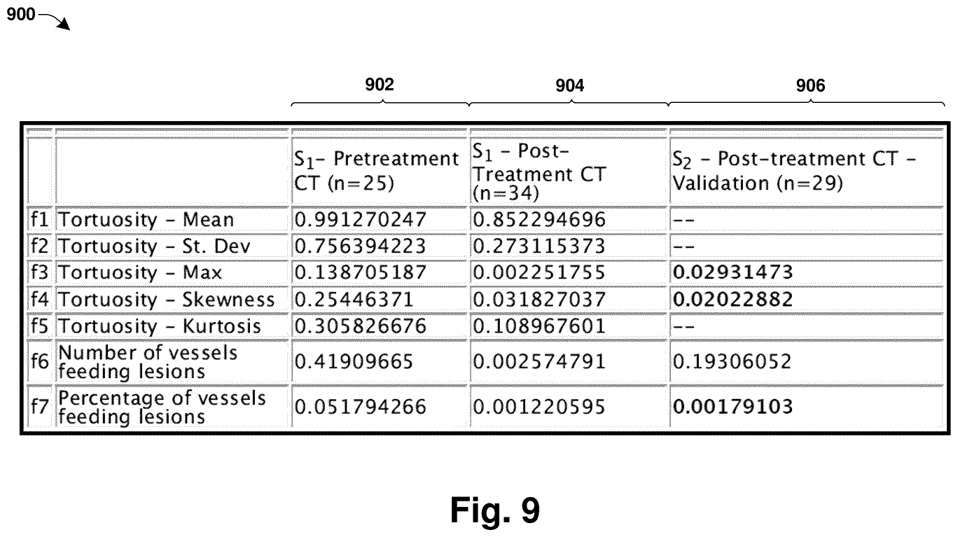

FIG. 9 illustrates a table 900 showing exemplary potential vascular radiology features that may be extracted during training of a disclosed machine learning model. Table 900 shows p-values for each of the potential vascular radiology features. In some embodiments, selected ones of the potential vascular radiology features having p-values that are less than 0.05 are determined to be important in determining overall survival of a patient.

As shown in table 900, seven potential vascular radiology features were identified. The seven potential vascular radiology features are a mean of a vessel tortuosity separately computed for each branch, a standard deviation of the vessel tortuosity computed for each branch, a maximum of the vessel tortuosity computed for each branch, skewness of the vessel tortuosity computed for each branch, a kurtosis of the vessel tortuosity computed for each branch, a number of hepatic blood vessels arising from a lesion, and a percentage of hepatic blood vessels arising from the lesion.

As shown in columns 902-904, within a training set (S₁) four of the seven potential vascular radiology features were found to be significantly associated with disease progression. The four features were tumor vascularization features (f6—HR=1.115 [1.039-1.196]; f7—HR=10.646 [2.539-44.641]), as well as two features of vessel tortuosity (f3—HR=0.011 [0.001-0.199]; f4—HR=0.545 [0.313-0.949]).

As shown in column 906, within the validation set (S₂), three of the seven potential vascular radiology features were found to be significantly associated with overall survival. The three features included the maximum of the vessel tortuosity computed for each branch (f3—HR=0.085 [0.009-0.780]), the skewness of the vessel tortuosity computed for each branch (f4—HR=0.331 [0.130-0.842]), and the percentage of vessels feeding the lesions (f7—HR=6.445 [2.001-20.753]]). However, the number of vessels feeding the lesions (f6—HR=1.070 [0.966-1.184]) was not found to be significantly associated with overall survival.

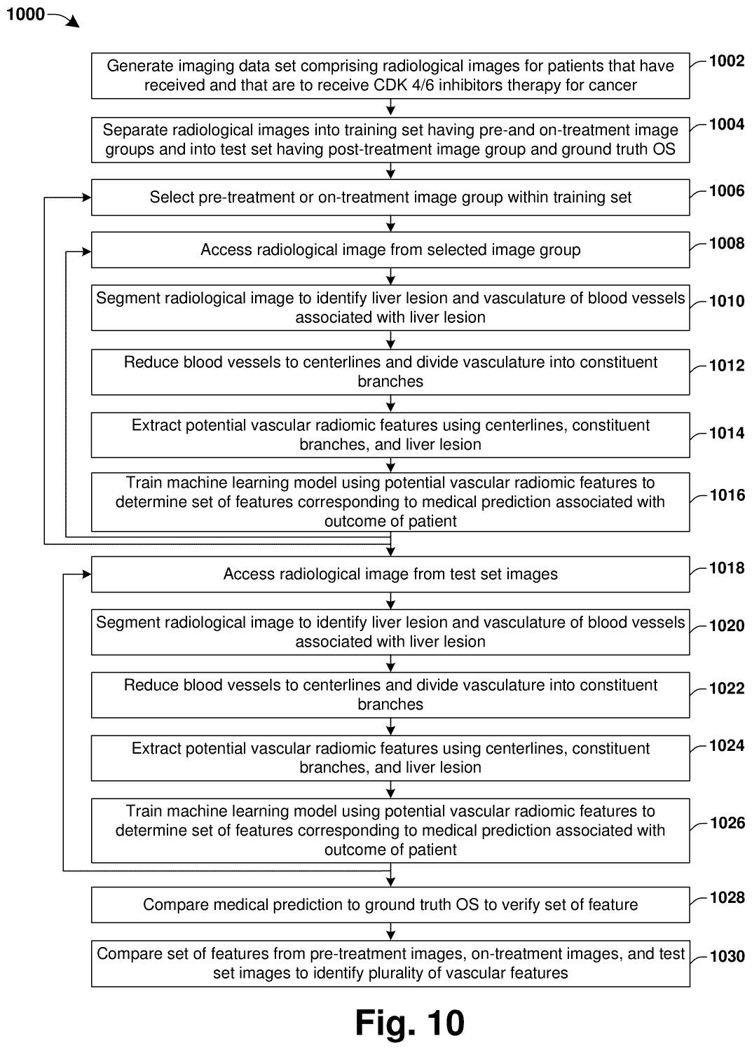

FIG. 10 illustrates some embodiments of a flow diagram showing a method 1000 for generating a machine learning model that uses vascular radiology features computationally extracted from radiological images to generate a medical prediction.

At act 1002, an imaging data set comprising radiological images for patients that have received and/or that are to receive CDK 4/6 inhibitors therapy for cancer (e.g., MBC) is generated.

At act 1004, the radiological images within the imaging data set are separated into a training set having pre- and on-treatment image groups and into a test set having an on-treatment image group and ground truth overall survival (OS) information.

At act 1006, a pre-treatment image group or an on-treatment image group is selected from within the training set.

At act 1008, a radiological image is accessed from a selected image group (e.g., from the pre-treatment image group or the on-treatment image group).

At act 1010, the radiological image is segmented to identify liver lesion and vasculature of blood vessels associated with the liver lesion.

At act 1012, the blood vessels are reduced to centerlines and the vasculature are divided into constituent branches.

At act 1014, potential vascular radiology features are extracted using the centerlines, the constituent branches, and/or the liver lesion.

At act 1016, a machine learning model is trained using the potential vascular radiology features to determine a set of features corresponding to a medical prediction associated with an outcome of a patient.

In some embodiments, after performing act 1016 the method may return to act 1006 to select a different image group. For example, in some embodiments the method 1000 may perform acts 1008-1016 on the pre-treatment image group and then subsequently perform acts 1008-1016 on the on-treatment image group. Furthermore, acts 1008-1016 may be iteratively performed over a plurality of radiological images within a selected image group. For example, acts 1008-1016 may be iteratively performed for respective ones of the plurality of pre-treatment images to determine a first set of features and then acts 1008-1016 may be iteratively performed for respective ones of the plurality of on-treatment images to determine a second set of features.

At act 1018, a radiological image is accessed from the test set images.

At act 1020, the radiological image is segmented to identify liver lesion and vasculature of blood vessels associated with the liver lesion.

At act 1022, the blood vessels are reduced to centerlines and the vasculature are divided into constituent branches.

At act 1024, potential vascular radiology features are extracted using the centerlines, the constituent branches, and/or the liver lesion.

At act 1026, a machine learning model is trained using the potential vascular radiology features to determine a set of features corresponding to a medical prediction associated with an outcome of a patient.

At act 1028, the medical prediction is compared to a ground truth overall survival (OS) to verify the set of features.

At act 1030, the set of features from the pre-treatment images within the training set, the on-treatment images within the training set, and the test set images are compared to identify a plurality of vascular features. The plurality of vascular features may be subsequently extracted from an additional patient (e.g., corresponding to the method 200 of FIG. 2) during a medical examination of the additional patient.

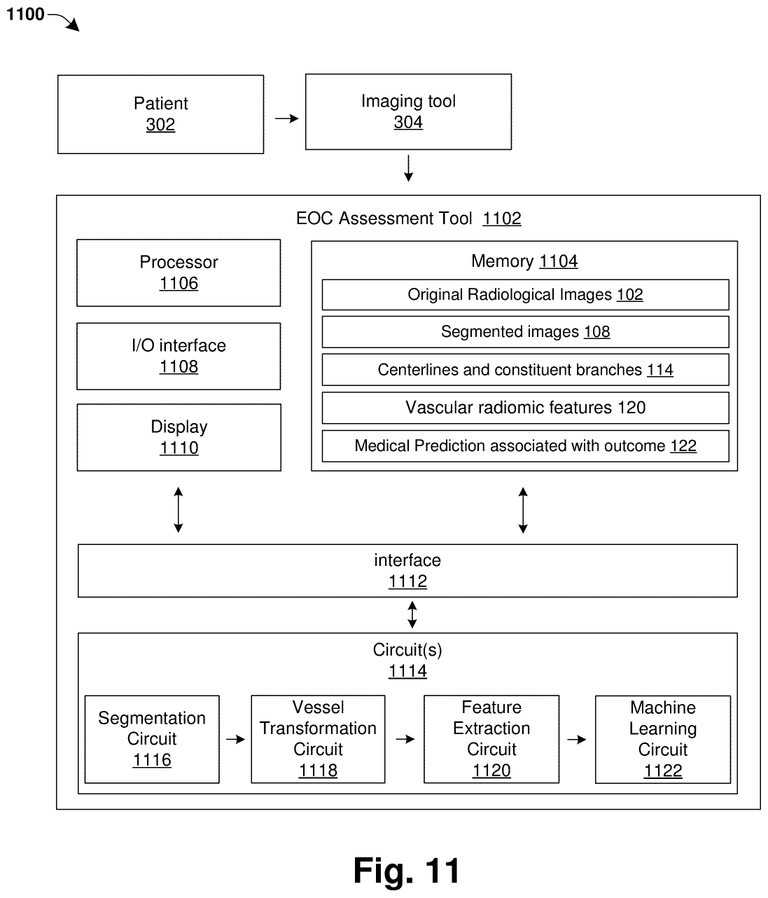

FIG. 11 illustrates a block diagram of some embodiments of a prognostic apparatus 1100 comprising a disclosed cancer assessment system.

The prognostic apparatus 1100 comprises a cancer assessment tool 1102. The cancer assessment tool 1102 is coupled to one or more imaging tools 304 (e.g., one or more radiological imaging devices) that are configured to be operated upon a patient 302.

The cancer assessment tool 1102 comprises a processor 1106 and a memory 1104. The processor 1106 can, in various embodiments, comprise circuitry such as, but not limited to, one or more single-core or multi-core processors. The processor 1106 can include any combination of general-purpose processors and dedicated processors (e.g., graphics processors, application processors, etc.). The processor 1106 can be coupled with and/or can comprise memory (e.g., memory 1104) or storage and can be configured to execute instructions stored in the memory 1104 or storage to enable various apparatus, applications, or operating systems to perform operations and/or methods discussed herein.

Memory 1104 can be further configured to store an imaging data set 102 comprising the one or more radiological images 104. The one or more radiological images 104 comprise digital images having a plurality of imaging units (e.g., pixels, voxels, etc.) respectively having an associated intensity pixels. The one or more radiological images 104 may comprise images that include a liver of a patient. In some additional embodiments, the one or more radiological images 104 may be stored in the memory 1104 as one or more training sets, test sets, and/or validation sets for training a machine learning circuit.

The cancer assessment tool 1102 also comprises an input/output (1/O) interface 1108 (e.g., associated with one or more I/O devices), a display 1110, one or more circuits 1114, and an interface 1112 that connects the processor 1106, the memory 1104, the I/O interface 1108, the display 1110, and the one or more circuits 1114. The I/O interface 1108 can be configured to transfer data between the memory 1104, the processor 1106, the one or more circuits 1114, and external devices (e.g., the one or more imaging tools 304).

In some embodiments, the one or more circuits 1114 may comprise hardware components. In other embodiments, the one or more circuits 1114 may comprise software components. In such embodiments, the one or more circuits 1114 may execute code stored in the memory 1104. The one or more circuits 1114 can comprise a segmentation circuit 1116 configured to segment respective ones of the one or more radiological images 104 to generate one or more segmented images 108 that identify a lesion and a vasculature of blood vessels associated with the lesion. In some embodiments, the segmentation circuit 1116 may comprise a deep learning model/circuit.

In some additional embodiments, the one or more circuits 1114 may further comprise a vessel transformation circuit 1118 configured to reduce blood vessels within the segmented image 108 to centerlines and to divide the vasculature within the segmented image 108 into constituent branches.

In some additional embodiments, the one or more circuits 1114 may further comprise a feature extraction circuit 1120. In some embodiments, the feature extraction circuit 1120 is configured to operate upon the centerlines and constituent branches 116, and/or the lesions 110 to extract a plurality of vascular radiology features 120 describing the blood vessels and/or the vasculature.

In some additional embodiments, the one or more circuits 1114 may further comprise a machine learning circuit 1122. In some embodiments, the machine learning circuit 1122 is configured to utilize the plurality of vascular radiology features 120 to generate a medical prediction associated with an outcome of a patient 122.

### Example Use Case

Method: From a registry of 350 patients on treatment with CDKI at institution 11(S), 51 pts with HR+, Her2−, MBC patients with evidence of liver metastasis and disease progression (PFS) data were identified. 30 patients discontinued treatment due to progression or death, with a median time to progression of 195 days. Pre-treatment and first post-treatment CT exams were analyzed from 25 and 34 patients, respectively. Median time between scans was 128 days. To validate the prognostic value of our signature, a cohort of 29 patients with available OS data was identified from institution 2 (S₂).

A pre-trained deep learning model was applied to isolate liver metastases and vessels. Next, a fast marching algorithm was applied to reduce vessels to their centerlines and divide the vasculature into constituent branches. 7 quantitative metrics were computed measuring vascularity of metastases and 3-D shape of hepatic blood vessels. First, the number (f1) and percentage (f2) of hepatic blood vessels arising from the tumor were computed. Vessel tortuosity—measuring the degree of twisting across a vessel—was computed separately for each branch. The mean (f3), standard deviation (f4), maximum (f5), skewness (f6), and kurtosis (f7) tortuosity values were calculated to summarize these measurements at patient level. The features were individually assessed at pre- and post-treatment for association with PFS at S₁ in univariable Cox proportional hazards models. Features found to be associated in1 S were evaluated for association with OS in S₂.

Results: On the initial post-treatment scan, features of both tumor vascularization (f6—HR=1.115 [1.039-1.196]; f7—HR=10.646 [2.539-44.641]), as well as two features of vessel tortuosity (f3—HR=0.011 [0.001-0.199]; f4—HR=0.545 [0.313-0.949]) were significantly associated with PFS. Both tortuosity features were also significantly associated with OS in S₂ (f3—HR=0.085 [0.009-0.780]; f4—HR=0.331 [0.130-0.842]). In addition, the percentage (f7—HR=6.445 [2.001-20.753]]), of vessels feeding the lesions was also significant in S₂ while the number (f6—HR=1.070 [0.966-1.184]), of vessels was not No vessel metrics from the pre-treatment baseline exam were significantly associated with OS.

Conclusions: Radiomic analysis of tumor vascularity and vessel tortuosity on CT scans post-CDK treatment was associated with patient survival and treatment response.

Therefore, the present disclosure relates to a method and associated apparatus for determining a medical prediction associated with an outcome of a patient using vascular radiology features extracted from radiological images of an organ having one or more metastases. The disclosed method and associated apparatus are able to achieve an improved performance over traditional manual assessments because the vascular radiology features that the disclosed method and apparatus extracts from the radiological images are at a higher order or higher level than a human can resolve in the human mind or with pencil and paper.

In some embodiments, the present disclosure relates to a method. The method includes accessing data including one or more segmented images identifying one or more lesions and/or a plurality of blood vessels associated with the one or more lesions, respective ones of the plurality of blood vessels corresponding to one or more centerlines and one or more constituent branches associated with the one or more lesions, the one or more segmented images being derived from one or more radiological images of a patient having cancer; extracting one or more vascular radiology features using the centerlines, the constituent branches, and the one or more lesions, the one or more vascular radiology features relating to a quantification of the plurality of blood vessels or a shape of the plurality of blood vessels; and using the one or more vascular radiology features to determine a medical prediction associated with an outcome of the patient to cyclin-dependent kinase (CDK) inhibitor therapy.

In other embodiments, the present disclosure relates to a non-transitory computer-readable medium storing computer-executable instructions that, when executed, cause a processor to perform operations, including accessing one or more segmented images of one or more computerized tomography (CT) scan images for a patient that has received or may receive a cyclin-dependent kinase 4 and 6 (CDK 4/6) inhibitor treatment for cancer, the one or more segmented images identifying one or more lesions and a vasculature of a plurality of hepatic blood vessels associated with the one or more lesions; extracting one or more vascular radiology features associated with the one or more lesions and the vasculature of the plurality of hepatic blood vessels, the one or more vascular radiology features relating to a quantification of the plurality of hepatic blood vessels and a tortuosity of the plurality of hepatic blood vessels; and using the one or more vascular radiology features to determine a medical prediction associated with an outcome of the patient.

In yet other embodiments, the present disclosure relates to an apparatus. The apparatus includes a memory configured to store one or more segmented images derived from one or more radiological images of a patient that has received or may receive a cyclin-dependent kinase (CDK) inhibitor treatment for cancer, the segmented image identifying one or more lesions and a vasculature of a plurality of blood vessels associated with the one or more lesions within a radiological image of the patient; a vessel transformation tool configured to reduce the plurality of blood vessels to centerlines and dividing the vasculature of the plurality of blood vessels into a plurality of constituent branches; a feature extraction tool configured to extract one or more vascular radiology features using the centerlines, the constituent branches, and the one or more lesions, the one or more vascular radiology features relating to a quantification of the plurality of blood vessels and a tortuosity of the plurality of blood vessels; and a machine learning model configured to operate upon the one or more vascular radiology features to determine a medical prediction associated with an outcome of the patient.

Examples herein can include subject matter such as an apparatus, including a digital whole slide scanner, a CT system, an MRI system, a personalized medicine system, a CADx system, a processor, a system, circuitry, a method, means for performing acts, steps, or blocks of the method, at least one machine-readable medium including executable instructions that, when performed by a machine (e.g., a processor with memory, an application-specific integrated circuit (ASIC), afield programmable gate array (FPGA), or the like) cause the machine to perform acts of the method or of an apparatus or system, according to embodiments and examples described.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

“Computer-readable storage device”, as used herein, refers to a device that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, and other media from which a computer, a processor or other electronic device can read.

“Circuit”, as used herein, includes but is not limited to hardware, firmware, software in execution on a machine, or combinations of each to perform a function(s) or an action(s), or to cause a function or action from another logic, method, or system. A circuit may include a software controlled microprocessor, a discrete logic (e.g., ASIC), an analog circuit, a digital circuit, a programmed logic device, a memory device containing instructions, and other physical devices. A circuit may include one or more gates, combinations of gates, or other circuit components. Where multiple logical circuits are described, it may be possible to incorporate the multiple logical circuits into one physical circuit. Similarly, where a single logical circuit is described, it may be possible to distribute that single logical circuit between multiple physical circuits.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A method, comprising:
accessing data including one or more segmented images identifying one or more lesions and/or a plurality of blood vessels associated with the one or more lesions, respective ones of the plurality of blood vessels corresponding to one or more centerlines and one or more constituent branches associated with the one or more lesions, wherein the one or more segmented images are derived from one or more radiological images of a patient having cancer;
extracting one or more vascular radiology features using the centerlines, the constituent branches, and the one or more lesions, wherein the one or more vascular radiology features relate to a quantification of the plurality of blood vessels or a shape of the plurality of blood vessels; and
using the one or more vascular radiology features to determine a medical prediction associated with an outcome of the patient to cyclin-dependent kinase (CDK) inhibitor therapy.

2. The method of claim 1, wherein the one or more vascular radiology features comprise one or more statistical measurements of a tortuosity of the plurality of blood vessels.

3. The method of claim 1, wherein the one or more vascular radiology features comprise a percentage of the plurality of blood vessels feeding the one or more lesions.

4. The method of claim 1, further comprising:
individually assessing the one or more vascular radiology features using pre-treatment and on-treatment images for association with the medical prediction associated with the outcome of the patient.

5. The method of claim 1, further comprising:
operating upon the one or more lesions and a vasculature of the plurality of blood vessels with a fast marching algorithm to reduce the plurality of blood vessels to the centerlines and to divide the vasculature of the plurality of blood vessels into the constituent branches.

6. The method of claim 1, wherein the patient has received and/or is receiving the CDK inhibitor therapy for metastatic breast cancer.

7. The method of claim 1, wherein the one or more lesions comprise a liver metastasis.

8. The method of claim 1, wherein the one or more radiological images are taken of the patient after initiation of treatment with the CDK inhibitor therapy.

9. The method of claim 1,
wherein the one or more radiological images include a pre-treatment radiological image taken of the patient before initiation of treatment with the CDK inhibitor therapy and an on-treatment radiological image taken of the patient after the initiation of treatment with the CDK inhibitor therapy; and
wherein the medical prediction associated with the outcome of the patient is determined based on the one or more vascular radiology features that correspond to both the pre-treatment radiological image and the on-treatment radiological image.

10. A non-transitory computer-readable medium storing computer-executable instructions that, when executed, cause a processor to perform operations, comprising:
accessing one or more segmented images of one or more computerized tomography (CT) scan images for a patient that has received or may receive a cyclin-dependent kinase 4 and 6 (CDK 4/6) inhibitor treatment for cancer, wherein the one or more segmented images identify one or more lesions and a vasculature of a plurality of hepatic blood vessels associated with the one or more lesions;
extracting one or more vascular radiology features associated with the one or more lesions and the vasculature of the plurality of hepatic blood vessels, wherein the one or more vascular radiology features relate to a quantification of the plurality of hepatic blood vessels and a tortuosity of the plurality of hepatic blood vessels; and
using the one or more vascular radiology features to determine a medical prediction associated with an outcome of the patient.

11. The non-transitory computer-readable medium of claim 10, further comprising:
reducing the plurality of hepatic blood vessels to a plurality of centerlines and dividing the vasculature of the plurality of hepatic blood vessels into a plurality of constituent branches; and
extracting the one or more vascular radiology features using the centerlines, the constituent branches, and the one or more lesions.

12. The non-transitory computer-readable medium of claim 10, wherein the one or more CT scan images are taken from the patient when the patient is enrolled or considered for enrollment in a treatment plan that calls for further CDK 4/6 inhibitor treatments.

13. The non-transitory computer-readable medium of claim 10, wherein the one or more CT scan images comprise one or more pre-treatment images and one or more on-treatment images of the patient.

14. The non-transitory computer-readable medium of claim 13, wherein the operations further include:
extracting a first group of the one or more vascular radiology features from a first segmented image derived from a pre-treatment radiological image;
extracting a second group of the one or more vascular radiology features from a second segmented image derived from an on-treatment radiological image; and
generating the medical prediction associated with the outcome of the patient using both the first group and the second group of the one or more vascular radiology features.

15. The non-transitory computer-readable medium of claim 10, wherein the operations further include:
measuring a tortuosity separately for respective ones of the plurality of hepatic blood vessels; and
performing a statistical measurement of the tortuosity measured separately for the respective ones of the plurality of hepatic blood vessels to generate the one or more vascular radiology features.

16. The non-transitory computer-readable medium of claim 10, wherein the one or more vascular radiology features comprise a percentage of the plurality of hepatic blood vessels feeding the one or more lesions, a maximum of a tortuosity of the plurality of hepatic blood vessels, and a skewness of a tortuosity of the plurality of hepatic blood vessels.

17. An apparatus, comprising:
a memory configured to store one or more segmented images derived from one or more radiological images of a patient that has received or may receive a cyclin-dependent kinase (CDK) inhibitor treatment for cancer, wherein the segmented image identifies one or more lesions and a vasculature of a plurality of blood vessels associated with the one or more lesions within a radiological image of the patient;
a vessel transformation tool configured to reduce the plurality of blood vessels to centerlines and dividing the vasculature of the plurality of blood vessels into a plurality of constituent branches;
a feature extraction tool configured to extract one or more vascular radiology features using the centerlines, the constituent branches, and the one or more lesions, wherein the one or more vascular radiology features relate to a quantification of the plurality of blood vessels and a tortuosity of the plurality of blood vessels; and
a machine learning model configured to operate upon the one or more vascular radiology features to determine a medical prediction associated with an outcome of the patient.

18. The apparatus of claim 17, wherein a vessel tortuosity is measured separately for respective ones of the plurality of constituent branches, wherein the vessel tortuosity is used to generate the one or more vascular radiology features.

19. The apparatus of claim 18, wherein a statistical assessment of the vessel tortuosity is separately performed for respective ones of the plurality of constituent branches.

20. The apparatus of claim 17, wherein the one or more lesions comprise a liver metastasis.
